(19) United States
(12) Patent Application Publication
(10) Pub. No.: US 2022/0202748 A9
(43) Pub. Date: Jun. 30, 2022

(54) SCALP COOLING HAIR PRODUCT

(71) Applicant: Sherlanda Fulks, Americus, GA (US)

(72) Inventor: Sherlanda Fulks, Americus, GA (US)

(21) Appl. No.: 16/855,385

(22) Filed: Apr. 22, 2020

Related U.S. Application Data
(60) Provisional application No. 62/837,850, filed on Apr. 24, 2019.

(51) Int. Cl. A61K 31/167; A61K 47/44; A61P 17/04

(52) U.S. Cl. CPC A61K 31/167; A61P 17/04; A61K 47/44

(57) ABSTRACT

The present invention relates generally to new and improved compositions and methods for producing reliable relief from irritation or itching of the skin of the scalp.

### FIELD OF THE INVENTION

The present invention relates generally to new and improved compositions and methods for producing reliable relief from irritation or itching of the skin of the scalp.

### BACKGROUND OF THE INVENTION

There are many substances that when applied to the scalp, can produce irritation or itching. Indeed, even environmental exposures (sun, heat, cold, humidity, pollutants) can produce a biological change in the skin of the scalp to produce irritation or itching. Mere washing with soaps, detergents, shampoos or other hair products can similarly cause the scalp to be irritated or to itch. Exposure of the scalp to topical products such as creams, gels, pastes, sprays or mists, lotions, moisturizers, fragrances, depilatories, shaving products, hair removal products; skin cleansers, toners and astringents; conditioners, colorants, dyes. bleaches, straighteners, and permanent wave products can all cause irritation of one form or another to the scalp.

“Irritation” can include various inflammation symptoms or signs when the hair or the skin of the scalp is exposed to such products or the environment. The severity of irritation to the susceptible user may range from subclinical to mild to severe. Typical symptoms of “irritation” include itching (pruritus), stinging, burning, tingling, “tightness,” erythema (redness) or edema (swelling). The irritation response may be due to the direct effect on the skin of certain topical product chemicals or to a response by the immune system directed toward the chemicals alone or in combination with skin components (e.g. antigens).

The sensation of itch is one of the most common scalp problems experienced by humans and animals. Itch can be defined as a sensation which provokes the desire to scratch the site from which the sensation originates. All skin, including that of the scalp, contains sensory nerves which can transmit itch or other sensory impulses in response to chemical irritation, environmental exposure or disease processes. It appears that the thinnest, unmyelinated nerve population, termed type C nociceptive neurons are thought to be the most important in producing the sensation. See for example: Itch: Mechanisms and Management of Pruritus. Jeffrey D. Bernhard. McGraw-Hill, Inc. (San Francisco, 1994), pp. 1-22. The sensory nerves of the skin can be considered to be a “final common pathway” for the many irritating conditions which may be ultimately sensed as itch including chemical exposure, environmental exposure (such as that which produces dry, itchy skin) and disease processes such as atopic dermatitis. Many chemical substances are able to produce itch or other sensory impulses when topically applied to the skin or scalp. No matter what the ultimate cause of itch, the sensation experienced is the same and provokes the desire to scratch.

Many ingredients used in topical products are known irritants or are potentially irritating, especially to people with “sensitive skin”. These irritating ingredients include fragrances, preservatives, solvents, propellants and many other ingredients that might otherwise be considered inert components of the products. Additionally, many topical product active ingredients, including chemicals that may also be classified as drugs, produce irritation when applied to the skin or scalp. These include, but are not limited to, such ingredients as exfoliants and skin cell renewal agents, anti-acne drugs, antiperspirant compounds, antihistamines, anti-inflammatory agents, skin protective agents, insect repellent chemicals, sunscreens and many others. Where more than one chemical irritant is present, their irritating effects may be additive. Furthermore, chemical ingredients may react with one another, or in the environment of the skin or scalp, to form new chemicals which are irritating. The vehicles in which the active drug ingredients are formulated may also produce irritation in sensitive people, especially in the case of drugs such as topical corticosteroids.

In addition to chemicals which directly trigger skin or scalp irritation, some chemicals indirectly cause the skin to become more sensitive to other chemicals or environmental conditions which would not normally cause irritation. Many chemicals which act as skin “exfoliants” may cause the skin to become more sensitive to irritation triggered by other topically-applied chemicals such as moisturizers, sunscreens, fragrances, preservatives, surfactants (e.g. soaps, shaving cream) and other topical products. Exfoliants and other ingredients may also increase the skin or scalp's sensitivity to environmental conditions such as sunlight, wind, cold temperature and dry air, or to chemical agents such as antigens, or may exacerbate the irritation attributable to a pre-existing skin disease.

Environmental influences may also increase the sensitivity of the skin or scalp to chemicals in topical products by reducing the epidermal skin's “barrier function.” The barrier function acts to minimize absorption or passage of potentially irritating chemicals through the outer “dead” cell layer of epidermal skin into the living skin tissue. Extremes of humidity, for example, can greatly increase irritation from topically-applied products. A very common condition due to low humidity is termed “winter itch” in which the very low humidity characteristics of many cold climates (particularly when accompanied by indoor heating) or long exposure to refrigerated air from air conditioners in the summer produces itchy skin or scalp which can exacerbate the irritating effects of topical products. Additionally, soaps, detergents, cleansing products, shaving creams, alcohol and other products which remove some of the skin's protective lipids and/or secretions may increase the skin's permeability and sensitivity to topically-applied chemicals which would otherwise not produce irritation.

Exposure of the skin or scalp to high humidity environments (eg. wearing of hats, or wigs) may also increase the ability of potential irritants to penetrate the skin or scalp. Similarly, the skin or scalp may become sensitized or inflamed due to shaving abrasion, repeated or excessive washing or bathing, sun exposure, or other mechanical abrasion or injury, resulting in sensory irritation responses upon subsequent application of a topical products.

There are also inherent sensitivities or genetic predispositions to skin irritants. Allergies, atopic dermatitis, allergic or non-allergic contact dermatitis, psoriasis, eczema, post-herpetic neuralgia, skin lesions such as candidiasis, insect bites and the like can produce intrinsic irritation which may be exacerbated by application of topical products or by exposure to chemical or environmental influences such as antigens, cold air, low humidity and the like. Many other individuals exhibit sensitive skin as a condition that is not related to an identifiable skin disease.

While there are many products advertised to consumers as “hypoallergenic,” many skin or scalp irritation responses do not respond to these products. Furthermore, shampoos or conditioners that are marketed for such purposes, may even exacerbate the scalp irritations or itch.

A need therefore exists in the art for new and improved compositions and methods for producing reliable relief from irritation or itching of the skin of the scalp. More particularly, a need exists in the art for new and improved compositions and methods that involve a “leave-in” product that can be left on the scalp without the need for rinsing or washing for several hours.

### SUMMARY OF THE INVENTION

The present invention relates generally to new and improved compositions and methods for producing reliable relief from irritation or itching of the skin of the scalp. More particularly, the new and improved compositions and methods involve an optional “leave-in” product that can be left on the scalp without the need for rinsing or washing for several hours. The compositions of the present invention can include one or more anesthetics or numbing agents, water, and optionally one or more of a carrier oil, an essential oil, an emulsifier, and a fragrance or aroma-enhancing agent. The compositions of the present invention can be formulated into sprays, mists or liquids for easy application and use.

These and other objects, along with advantages and features of the embodiments of the present invention herein disclosed, will become more apparent through reference to the following description, the examples, and the claims. Furthermore, it is to be understood that the features of the various embodiments described herein are not mutually exclusive and can exist in various combinations and permutations.

### DETAILED DESCRIPTION OF THE INVENTION

Various other features and attendant advantages of the present invention will be more fully appreciated from the following detailed description.

The scalp cosmetic compositions and corresponding methods of the present invention are directed to reducing itching or irritation of the scalp. The compositions can include one or more one or more anesthetics or numbing agents, water, and optionally one or more of a carrier oil, an essential oil, an emulsifier, and a fragrance or odor-enhancing agent.

Each of these ingredients will be described in more detail below. The compositions described herein can be utilized as “leave-on” or wash and/or rinse off.

The scalp cosmetic compositions of the present invention can be formulated into sprays, mists, gels, creams or liquids for easy application and use. The compositions of the present invention can be applied by hand, with a spray bottle or mister, or by applicator. Optionally, the compositions of the present invention can be incorporated into another product such as shampoos, conditioners, styling products and the like.

The term “leave-on” refers to any of compositions of the present invention where the composition is applied to the desired area of the skin or scalp without any subsequently rinsing or shampooing the applied area for a period of at least about 30 min to 12 hours after application. The term “leave-on” specifically excludes any such compositions that is intended to be applied to the skin or scalp and then rinsed immediately or within several minutes. In some embodiments, the composition can of the present invention can applied without subsequent rinsing for at least 1 hour, for at least 2 hours, for at least 3, hours, for at least 4 hours, for at least 5 hours, for at least 6 hours, for at least 7 hours, for at least 8 hours, for at least 9 hours, for at least 10 hours, for at least 11 hours, or for at least 12 hours. Such compositions include shampoos, skin cleansing compositions, and the like.

The terms “rinse” or “washed off” indicates that the product is removed with water, i.e. rinsed or washed after a few minutes following application. In some embodiments the product is removed with purified water, tap water, mineral water, or scented water.

The scalp cosmetic compositions of the present invention are applied directly to the scalp and can be utilized to reduce itching and/or irritation to the skin. The “direct application” means that the composition can, for example, be applied to the hand and them rubbed or massaged onto the scalp, rather than to the hair. The composition can also be applied with an applicator or other packaging system.

The scalp cosmetic compositions and corresponding methods of the present invention can comprise, consist of, or consist essentially of the essential elements and limitations of the invention described herein, as well as any of the additional or optional ingredients, components, or limitations described herein or otherwise effective for such use.

### Topical Anesthetic or Numbing Agent

The topical anesthetic or numbing agent can be any local anesthetic agent including but not limited to lidocaine (0.1-20%); prilocaine (0.1-10%), tetracaine (0.1-10%), benzocaine (0.1-20%), proparacaine (0.1-20%), butamben (0.1-20%), dibucaine (0.1-20%), oxybuprocaine (0.1-20%), pramoxine (0.1-20%), and proxymetacaine (0.1-20%), or mixtures thereof. The anesthetic or numbing agent can be in the form a gel, powder, or liquid in any type of organic or inorganic base material to facilitate solubility. Mixtures of the topical anesthetic or numbing agent can also be eutectic mixtures. Eutectic mixtures are compounds which melt at lower temperatures than any of their components and thus permit higher concentrations of anesthetics for use. Eutectic mixtures include in addition to the local anesthetic, an oil in water emulsion cream, a thickener, an emulsifier, and distilled water.

### Carrier Oil

The compositions of the present invention can also include an oil. Oils can include, but are not limited to one or more of a carrier oil such as any form or grade of: Abyssinian, almond, aloe vera, apricot kernel, argan, avocado, babassu, baobab, black cumin seed, black currant seed, borage, broccoli seed, camellia seed, canola, carrot (macerated), castor, cherry kernel, chia seed, chili seed, coconut, evening primrose, flax seed, grapeseed, hazelnut, hemp seed, jojoba, kukui nut, macadamia, manila, meadowfoam, moringa, neem, oat, olive, palm, peach kernel, perilla seed, pomegranate, pracaxi, prickly pear, pumpkin seed, pumpkin seed, raspberry seed, raspberry seed, ricebran, rosehip, sacha inchi, safflower, sea buckthorn, sesame, soya bean, sunflower, tamanu, walnut, and wheat germ.

### Essential Oil

The compositions of the current invention can also include one or more of an essential oil, in any form or grade, such as: agarwood, ajwain (Carum copticum), Angelica root oil (distilled from the Angelica archangelica), anise oil, asafoetida oil, balsam, basil oil, bay oil, bergamot oil, black pepper oil, buchu oil, birch oil, camphor oil, cannabis flower oil, calamodin oil, caraway seed oil, cardamom seed oil, carrot seed oil, cedar oil, chamomile oil, calamus oil, cinnamon oil, citron oil, citronella oil, clary sage oil, coconut oil, clove oil, coffee oil, coriander oil, costmary oil, costus root oil, cranberry seed oil, cubeb oil, cumin seed oil, cypress oil, cypriol oil, curry leaf oil, davana oil, dill oil, elecampane oil, elemi oil, eucalyptus oil, fennel seed oil, fenugreek oil, fir oil, frankincense oil, galangal oil, galbanum oil, garlic oil, geranium oil, ginger oil, goldenrod oil, grapefruit oil, henna oil, helichrysum oil, hickory nut oil, horseradish oil, hyssop, jasmine oil, juniper berry oil, Laurus nobilis, lavender oil, ledum, lemon oil, lemon balm, lemongrass, lime, Litsea cubeba oil, linalool, limonene, mandarin, marjoram, melissa oil, Mentha arvensis oil, mint oil, moringa oil, mountain savory, mugwort oil, mustard oil, myrrh oil, myrtle, neem oil, neemt ree oil, neroli oil, nutmeg oil, orange oil, orris oil, palo santo, parsley oil, patchouli oil, perilla essential oil, peppermint oil, petitgrain, pine oil, ravensara, red cedar, Roman chamomile, rose oil, rosehip oil, rosemary oil, rosewood oil, sage oil, star anise oil, sandalwood oil, sassafras oil, savory oil, schisandra oil, spearmint oil, spikenard, spruce oil, tangerine, tarragon oil, tea tree oil, thyme oil, tsuga, turmeric, Valerian warionia, vetiver oil (khus oil), western red cedar, wintergreen, yarrow oil, ylang-ylang and zedoary. See Lawless, J., “The Encyclopedia of Essential Oils: The Complete Guide to the Use of Aromatic Oils in Aromatherapy, Herbalism, Health, and Well Being,” Conanri Press, Inc, 2013, incorporated herein by reference.

### Emulsifying Agents

The compositions of the current invention can also include one or more of an emulsifying agent, in any grade or form, including but not limited to: milk, yogurt, honey, aloe vera gel, soap, alcohol, gelatin, salts, witch hazel, vinegar, cornstarch, bentonite clay, egg yolk, lecithin, mustard, lecithins, sodium phosphates, monoglycerides, diglycerides, sodium stearoyl lactylate, diacetyl tartaric acid ester of monoglyceride, cellulose, detergents, emulsifying wax, polysorbate 20, ceteareth 20, polysorbate 80, polysorbate 40, polysorbate 60, polysorbate 65, ammonium phosphatides, sodium, potassium, magnesium and calcium salts of fatty acids, mono- and diglycerides of fatty acids, acetic acid esters of mono- and diglycerides of fatty acids, lactic acid esters of mono- and diglycerides of fatty acids, citric acid esters of mono- and diglycerides of fatty acids, mono- and diacetyl tartaric acid esters of mono- and diglycerides of fatty acids, mixed acetic and tartaric acid esters of mono- and diglycerides of fatty acids, sucrose esters of fatty acids, sucroglycerides, polyglycerol esters of fatty acids, polyglycerol polyricinoleate, propane-1,2-diol esters of fatty acids, thermally oxidized soya bean oil interacted with mono- and diglycerides of fatty acids, sodium stearoyl-2-lactylate, calcium stearoyl-2-lactylate, sorbitan monostearate, sorbitan tristearate, sorbitan monolaurate, sorbitan monooleate, and sorbitan monopalmitate.

### Other Ingredients

The compositions of the current invention can also include varying amounts of water, as well as one or more of a fragrance or aroma enhancing agents.

### EXAMPLES

### Example 1

Benzocaine gel (20% for dental use, bubblegum flavor, Quala Dental Products, 1 teaspoon) was blended with: i) organic aloe gel (harvested fresh from the aloe leaf, two leaves from a small aloe plant); ii) organic olive oil (Organic Great Value First Cold Pressed Extra Virgin Olive Oil, 3 drops; iii) organic aloe juice (liquid; Organic Lily of the desert Preservative Free Aloe Vera Juice Inner Fillet, 8 ml). The benzocaine gel was first mixed with 8 ml of Organic Lily of the desert Preservative Free Aloe Vera Juice Inner Fillet to loosen the hard gel first, then all of the other ingredients were measured and placed into a 10 ml spray bottle. Mixing was accomplished by slowly shaking. Product was applied (sprayed) onto the scalp. Finished product contained white flakes in the mixtures and following application onto the scalp, dried with the white flakes visible.

### Example 2

The same material and method as Example 1 except that distilled water (8 ml) was substituted for organic aloe juice. Finished product contained white flakes in the mixtures and following application onto the scalp, dried with the white flakes visible.

### Example 3

Lidocaine powder (5%; Spec-Pure, 10 grams) is blended with: i) organic aloe gel (harvested fresh from the leaf; harvested fresh from the aloe leaf, two leaves from a small aloe plant); ii) organic olive oil (Organic Great Value First Cold Pressed Extra Virgin Olive Oil, 3 drops) iii) organic aloe juice (liquid; Organic Lily of the desert Preservative Free Aloe Vera Juice Inner Fillet, 8 ml.) All ingredients were measured and placed into a 10 ml spray bottle. Mixing was accomplished by slowly shaking. Product was applied (sprayed) onto the scalp. This combination provided minimal numbing properties following application to the scalp. Finished product dried leaving a white powdery film on the scalp.

### Example 4

The same material and method as Example 3 except that distilled water (8 ml) was substituted for organic aloe juice. This combination provided minimal numbing properties following application to the scalp. Finished product dried leaving a white powdery film on the scalp.

### Example 5

Benzocaine gel (20% for dental use, bubblegum flavor, Quala Dental Products, 1 teaspoon was blended with organic olive oil (Organic Great Value First Cold Pressed Extra Virgin Olive Oil, 5 ml). All ingredients were measured and placed into a 10 ml spray bottle. Mixing was accomplished by slowly shaking. Ingredients did not mix well and they were too heavy to spray correctly from the 10 ml spray bottle.

### Example 6

Lidocaine gel (5% powder, Spec-Pure, 10 grams) was blended with organic olive oil (Organic Great Value First Cold Pressed Extra Virgin Olive Oil, 5 ml). All ingredients were measured and placed into a 10 ml spray bottle. Mixing was accomplished by slowly shaking. Ingredients did not mix well and they were too heavy to spray correctly from the 10 ml spray bottle.

### Example 7

Lidocaine (5% in a clear organic base, used for skin numbing during tattooing; 10 drops (SNG Labs) was blended with: i) aloe juice (liquid; Organic Lily of the Desert Preservative Free Aloe Vera Juice Inner Fillet 8 ml); ii) peppermint oil (Gurunanda 100% peppermint essential oil, 2 drops). All ingredients were measured and placed into a 10 ml spray bottle. Mixing was accomplished by slowly shaking. Product was applied (sprayed) onto the scalp. Finished product provided itching relief for up to 5 hrs.

### Example 8

Lidocaine (5% in a clear organic base, used for skin numbing during tattooing; 10 drops) was blended with: i) distilled water (8 ml); ii) peppermint oil (1 drop; Gurunanda 100% peppermint essential oil). All ingredients were measured and placed into a 10 ml spray bottle. Mixing was accomplished by slowly shaking. Product was applied (sprayed) onto the scalp. Finished product provided itching relief for up to 5 hrs.

### Example 9

Lidocaine (5% in a clear organic base, used for skin numbing during tattooing; 10 drops) was blended with distilled water (8 ml). All ingredients were measured and placed into a 10 ml spray bottle. Mixing was accomplished by slowly shaking. Product was applied (sprayed) onto the scalp. Finished product provided itching relief for up to 5 hrs.

### Example 10

The same material and method as Example 9, except with 5 drops of lidocaine (5%), distilled water (9 ml) and 1 drop of peppermint oil. This combination provided minimal numbing properties following application to the scalp. Finished product provided itching relief up to about 2.5 hours.

Other variations and modifications of the present invention are possible in light of the above teachings, and it is to be construed that such variations and modifications are effectively to be included within the claimed method and apparatus.

What is claimed is:

1. A composition for the treatment of irritation or itching of the scalp comprising a topical anesthetic, water and optionally at least one carrier oil.

2. The composition of claim 1 further comprising an essential oil.

3. The composition of claim 1 further comprising an emulsifier.

4. The composition of claim 1 further comprising a fragrance.

5. The composition of claim 1 formulated into a spray or mist.

6. The composition of claim 1, wherein the topical anesthetic is present at a concentration of about 0.1 to 20%.

7. A composition for the treatment of irritation or itching of the scalp comprising a topical anesthetic present at a concentration of about 0.1 to 20%, water, at least one carrier oil, at least one essential oil, an emulsifier and fragrance.

8. The composition of claim 7 formulated into a spray or mist.

9. A method for the treatment of irritation or itching of the scalp comprising directly applying a composition comprising a topical anesthetic, water and optionally at least one carrier oil.

10. The method of claim 9, wherein the composition further comprises an essential oil.

11. The method of claim 9, wherein the composition further comprises an emulsifier.

12. The method of claim 9, wherein the composition further comprises a fragrance.

13. The method of claim 9, where in the composition is applied by hand.

14. The method of claim 9, wherein the composition is massaged into the scalp.

15. The method of claim 1, wherein the composition is allowed to remain on the scalp for up to 5 hours.

16. The method of claim 1, wherein the composition is immediately rinsed off the scalp.

17. A method for the treatment of irritation or itching of the scalp comprising applying a composition comprising a topical anesthetic present at a concentration of about 0.1 to 20%, water, at least one carrier oil, at least one essential oil, an emulsifier and fragrance.

18. The method of claim 17, wherein the composition is massaged into the scalp.

19. The method of claim 17, wherein the composition is allowed to remain on the scalp for up to 5 hours.

20. The method of claim 17, wherein the composition is immediately rinsed off the scalp.
